(19) United States
(12) Patent Application Publication
(10) Pub. No.: US 2019/0079465 A1
(43) Pub. Date: Mar. 14, 2019

(54) REM Wake-up Alarm

(71) Applicant: Ali Ahmad, Plano, TX (US)

(72) Inventor: Ali Ahmad, Plano, TX (US)

(21) Appl. No.: 15/702,530

(22) Filed: Sep. 12, 2017

(51) Int. Cl. G04G 21/02; A61B 5/00; G08B 21/06; G04G 13/02

(52) U.S. Cl. CPC G04G 21/025; A61B 5/4812; G04G 13/021; G08B 21/06; A61B 5/746

(57) ABSTRACT

A Wake-up Alarm device that receives REM sleep data from Sleep Tracker Device and sets the Alarm On based on sleeping person's setting on REM sleep

Device that receives REM sleep data from sleep tracker device and waits until the REM sleep is over before starting wake-up alarm

Wake-up Alarm device is linked up with Sleep Tracker device like FitBit which monitors REM sleep using subject's heartbeat and body temperature, receives REM sleep start and end times and how deep REM sleep is and uses this data in an algorithm (software program) that decides whether to sound the wake-up alarm in order to provide most healthy sleep. If the subject has not have had enough REM sleep for the night and is in REM sleep at the alarm time then Wake-up Alarm device waits until the REM sleep is over. Also, if the subject is in deep REM sleep and subject set the Wake-up Alarm device to always wait for deep REM sleep to be over then the Wake-up Alarm device waits otherwise it starts the alarm and wakes up the subject. Block Diagram shows the Wake-up Alarm+Sleep Tracker system.

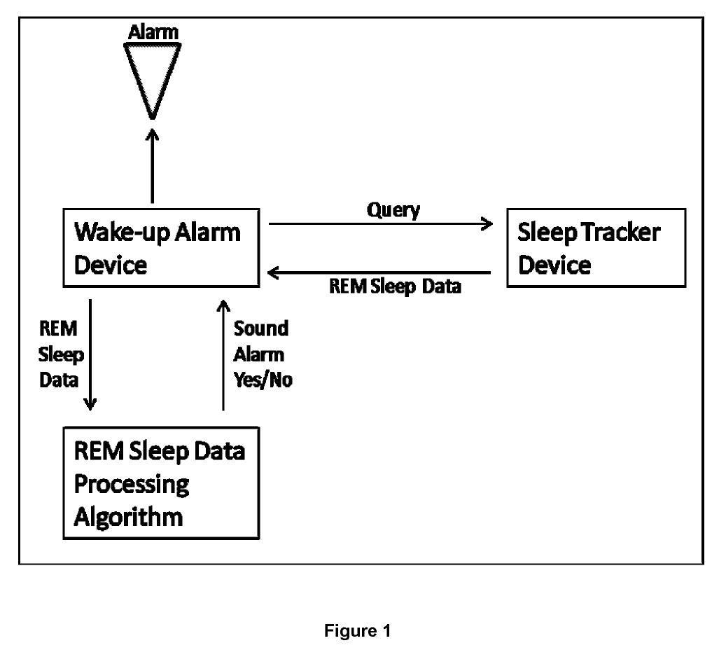

FIG. 1 in WakeUpAlarmUsingREM_Drawing.pdf document shows the block diagram for the REM Wake up Alarm system including wake up alarm device, REM Data processing algorithm and sleep tracker device. Diagram in FIG. 1 also shows the flow of data between parts of the system to achieve a properly timed wake up alarm.

1) Any Wake-up Alarm device that communicates to any sleep monitoring device.

2) Any Wake-up Alarm device that takes REM sleep monitoring data which includes start/stop of REM sleep and the depth level of REM sleep.

3) Any Wake-up Alarm device that takes REM sleep monitoring data runs it through an algorithm (a software program) which processes the REM sleep data for the most optimum healthy sleep.

4) In connection with claim #3, any algorithm that processes REM sleep data for the subject for the night and determines if subject had a good night sleep.

5) In connection with claim #3, any Algorithm that takes the REM sleep data and sends signal to Wake-up Alarm device if it is okay to wake-up the subject on the set alarm time or to wait until the REM sleep of the subject is over.

6) In connection with claim #3, any Wake-up Alarm device that takes the signal from algorithm (a software program) in the form of Yes or No to sound the Alarm

7) In connection with claim #2, any Wake-up Alarm device that has 3 settings on sounding the Alarm: 1) Wake me up anyway, 2) Wait until entire Deep REM sleep is over, 3) Wait until the peak of Deep REM sleep is over
